(19) United States
(12) Patent Application Publication
(10) Pub. No.: US 2017/0319723 A1
(43) Pub. Date: Nov. 9, 2017

(54) RADIOIMMUNE COMPLEX, THERANOSTIC AGENT AND KIT

(71) Applicant: Institute of Nuclear Energy Research, Atomic Energy Council, Executive Yuan, R.O.C., Taoyuan (TW)

(72) Inventors: WAN-I KUO, Taoyuan (TW); KAI-HUNG CHENG, Taoyuan (TW); YI-SHU HUANG, Taoyuan (TW); SHENG-NAN LO, Taoyuan (TW); YA-JEN CHANG, Taoyuan (TW); TSUNG-TSE WU, Taoyuan (TW); WEI-CHUAN HSU, Taoyuan (TW); MING-HSIN LI, Taoyuan (TW); CHIH-HSIEN CHANG, Taoyuan (TW)

(21) Appl. No.: 15/585,384

(22) Filed: May 3, 2017

(30) Foreign Application Priority Data
May 3, 2016 (TW) 105113746

(51) Int. Cl. A61K 51/10; A61K 51/04; A61K 47/22; C07K 16/28

(52) U.S. Cl. CPC A61K 51/103; A61K 51/1096; C07K 16/2863; A61K 47/22; A61K 51/0478; C07K 2317/24

(57) ABSTRACT

Disclosed herein is a radioimmune complex comprising an epidermal growth factor receptor (EGFR)-targeted antibody and a radioactive isotope of rhenium labeled thereon. The EGFR-targeted antibody is cetuximab or panitumumab.

### CROSS-REFERENCE TO RELATED APPLICATION

This application also claims priority to Taiwan Patent Application No. 105113746 filed in the Taiwan Patent Office on May 3, 2016, the entire content of which is incorporated herein by reference.

### TECHNICAL FIELD

The present disclosure relates to the technical field of radioimmunity, and more particularly to an antibody complex labeled with a radioactive isotope.

### BACKGROUND

In recent years, due to the aging of population, the westernized diet, and the increased intake of animal fat, the incidence of colorectal cancer rises year by year, and the age of onset also gradually declines. Based on the data from the Ministry of Health and Welfare, the standard human mortality of colorectal cancer (including colon and rectum) in 2014 is as high as 23.9 people in every 1,000,000 people.

According to the “Guidelines for clinical diagnosis and treatment of colorectal cancer” in 2014 of Taiwan Cooperative Oncology Group (TCOG) of National Health Research Institutes, multiple risk factors may cause the colorectal cancer. In addition to the environmental factors such as complex diet factor and physical activity, 15-30% of the incidence may be attributed to genetic heritage. Metastasis may occur to about 50-60% of the patients with colorectal cancer, primarily or non-primarily. The statistical data in medical science show that after receiving treatment, the five-year survival rate of the patients with stage I colorectal cancer is up to above 90%; the five-year survival rate of the patients with metastasis at the end of stage II is about 70%; the five-year survival rate of the patients with metastasis to lymph in stage III is about 50%; and the patients with remote metastasis in stage IV have the worst prognosis, and the five-year survival rate is only 5%. Therefore, if the colorectal cancer can be diagnosed in an early stage and effectively treated as early as possible, the survival rate of the patients can be increased greatly.

At present, for the treatment modalities for the patients with colorectal cancer and depending on different courses of disease, different therapeutic regimens may be given, for example, surgical operation, chemotherapy, radiation therapy or targeted therapy, and so on. The method for treating patients with metastatic colorectal cancer is mainly chemotherapy (e.g., fluoropyrimidine, oxaliplatin, and irinotecan), adjuvanted with targeted injection (e.g., Avastin or Erbitux). However, both the chemotherapy and the targeted therapy have a limited therapeutic effect, and the strong adverse effect also causes a high burden to the patients.

In addition, in order to provide a more accurate treatment for cancer patients, efforts are currently devoted to the development of “precision medicine” in the art, that is, an accurate level can be maintained from the early prevention, diagnosis, to the treatment stage. Therefore, personalized medicine has become a development focus in modern treatment and care of diseases, in which the products for molecular detection and diagnosis play a critical role. To improve the treatment accuracy and avoid the waste of medical resources, sensitive techniques such as immunoPET and immunoSPECT are used in combination with antibodies in the art, to accurately target the tumors, and rapidly screen out monoantibodies matching with the patients. However, the diagnostic agent that can be used in combination with immunoPET and immunoSPECT is limited, and cannot meet the clinical demand. Among the radioactive immune complex drugs developed, there is no Re-188 related radioimmune complex drug got available in the market at present. The production of nuclear medicines needs to be in conformity with the regulation of GMP (PIC/s GMP in Taiwan at present), and involves the production, and subsequent quality control and disposal of radioactive nuclide. The process for preparing the drugs is complex, and purification is required to achieve a radiochemical purity that is up to above 90%, whereby the drug cannot be directly used in hospitals. Accordingly, the clinical use or technology development (because the drug can be produced by only few manufacturers) of the drugs are directly affected. It is critical to develop a diagnostic or therapeutic agent containing radioactive antibodies in nuclear medicine, and prepare it into a kit that needs no purification and can be directly injected into patients, thereby reducing the radiation dose of the operator, facilitating the direct use in hospitals, and selling to various regions in the world. Moreover, Re-188 can emit γ ray useful in the diagnosis and β ray useful in the treatment, and has a half life of 16.9 hrs. The patients may be discharged from hospital after the activity drops to a background value, thereby improving the convenience in clinical use. An important object of the present patent is to produce a new radioimmune complex drug having diagnostic and therapeutic effects, and a method for preparing a kit containing the same is also provided. The method is progressive as a clinically feasible protocol for preparing a drug.

In view of this, there is an urgent need in the art for a composition, pharmaceutical product, or kit useful in cancer detection, diagnosis, and treatment, to overcome the defects in the prior art.

### SUMMARY

To make the essential meanings of the disclosure comprehensible to the reader, the summary provides the brief description of the disclosure. However, the summary is not elaboration of the disclosure, and not intended to define the technical features and the scope of the claims of the present invention.

To solve the above problems, this disclosure provides a new radioimmune complex useful in the treatment of cancers, which has a specific antibody (or a fragment thereof) and a radioactive isotope integrated, such that the radioactive isotope can be delivered to a target site by means of the specificity of antibody binding, thereby improving the therapeutic effect.

An aspect of the present invention relates to a radioimmune complex, comprising an epidermal growth factor receptor (EGFR)-targeted antibody and a radioactive isotope of rhenium. The EGFR-targeted antibody is cetuximab or panitumumab, and the radioactive isotope of rhenium is labeled on the antibody.

According to an optional embodiment, the radioactive isotope of rhenium is rhenium-188 or rhenium-186.

In a non-limiting embodiment, the EGFR-targeted antibody is reduced with 2-mercaptoethanol. According to a specific embodiment of this disclosure, the radioimmune complex of the present invention is prepared through a process comprising
a) treating the EGFR-targeted antibody with 2-mercaptoethanol, to obtain a reduced EGFR-targeted antibody;
b) adding a complexing agent to the reduced EGFR-targeted antibody; and
c) labeling the EGFR-targeted antibody with a radioactive isotope of rhenium.

According to an embodiment of this disclosure, in Step b), a reducing agent and stabilizing agent may be added to the reduced EGFR-targeted antibody. In an embodiment, the radioimmune complex prepared through the process above comprises cetuximab as the EGFR-targeted antibody, and in Step b), a complexing agent, a reducing agent, and a stabilizing agent need to be added to the reduced cetuximab.

In a specific embodiment, the complexing agent is methylene diphosphonate. In another embodiment, the reducing agent is stannous chloride. In another embodiment, the stabilizing agent is ascorbic acid.

Another aspect of this disclosure relates to a radioactive theranostic agent, comprising a theranostically effective amount of a radioimmune complex and a theranostically acceptable excipient. In an optional embodiment, the radioimmune complex is a radioimmune complex as shown in any one of the above embodiments.

Another aspect of this disclosure relates to a kit comprising an EGFR-targeted antibody, 2-mercaptoethanol, and a complexing agent. The EGFR-targeted antibody may be cetuximab or panitumumab, and the complexing agent is methylene diphosphonate.

In an embodiment, the kit further comprises a reducing agent and a stabilizing agent. Specifically, the reducing agent is stannous chloride, and the stabilizing agent is ascorbic acid.

The central concept and the employed technical means and various embodiments of the present invention may be fully understood by those of ordinary skill in the art to which this invention belongs upon reading the detailed description of the present invention.

### BRIEF DESCRIPTION OF DRAWINGS

To make the above and other objectives, features, advantages, and examples of the present invention more comprehensible, the drawings are illustrated below, in which

FIG. 1 is a radio-TLC scanning map of rhenium-188-panitumumab immune complex according to an embodiment of the present invention;

FIG. 2 shows a rhenium-188-cetuximab immune complex according to an embodiment of the present invention;

FIG. 3 is a nanoSPECT/CT image with rhenium-188-panitumumab in xenografted animal model of human lung cancer cells according to an embodiment of the present invention;

FIG. 4 is a nanoSPECT/CT image with rhenium-188-cetuximab in xenografted animal model of human lung cancer cells according to another embodiment of the present invention;

FIG. 5 is a bar diagram showing a binding activity of rhenium-188-cetuximab to A431 cells according to another embodiment of the present invention; and

FIG. 6 is a bar diagram showing the cytotoxic effect of rhenium-188 and cetuximab on human colorectal cancer cells according to another embodiment of the present invention.

### DETAILED DESCRIPTION OF DISCLOSED EMBODIMENTS

To make the description of the disclosure more thorough and complete, the implementations and specific examples of the present invention are exemplarily described below, which however, are not exhaustive.

Unless stated otherwise, the scientific and technical terms used herein have the same meanings as commonly understood and used by one of ordinary skill in the art. Furthermore, the noun used herein embraces both the singular and plural forms of the referents, unless indicated otherwise.

As used herein, the term “about” generally refers to that the actual value is within 10%, 5%, 1%, or 0.5% of a particular value or range. The term “about” means herein that the actual value is within an acceptable standard error of the mean, depending on the considerations of those ordinary skill in the art to which this invention belongs. Besides the experimental examples, or unless stated specifically otherwise, it should be understood that the ranges, amounts, numerical values, and percentages used herein are modified by “about”. Therefore, unless stated otherwise, the numerical values or parameters disclosed in the specification and claims are all rough values and may be varied as desired.

To overcome the defects of the conventional methods for treating cancers, an aspect of the present invention provides an immune complex for use in the field of radioimmunotherapy, in which a radioactive isotope of rhenium is directly labeled on the thiol group of cetuximab or panitumumab, to produce a new radioimmune complex, which is effective in the treatment and detection of cancers with high expression of EGFR, including, but not limited to, metastatic colorectal cancer, metastatic non-small lung cell cancer, and head and neck cancer.

In the radioimmune complex according to the present invention, the radioactive isotope of rhenium (e.g. rhenium-188) has a half-life of 16.9 hrs, and the experimental result shows that the image by nanoSPECT/CT is good, such that the metastatic cancer cells and the course of disease can be detected and the therapeutic effect can be evaluated non-invasively. Cetuximab or panitumumab is an EGFR specific antibody, such that the radioimmune complex of the present invention can effectively block the effect of EGFR, stop the growth of cancer cells, and kill cancer cells by radioactive isotope, thus having an additive therapeutic effect. In addition, the effect of the targeting radioimmune complex provided in the present invention on the immune system can be ignored.

Furthermore, for early diagnosis of cancers with high expression of EGFR, another aspect of the present invention relates to a radioactive theranostic agent, comprising a radioimmune complex and a theranostically acceptable excipient. The dosage of the theranostic agent dosed to a subject may be adjusted according to the physiological conditions and status of the subject and purpose of treatment, for example, the the course of disease, gender, or body weight of the patients. The effective dosage may be decided by those of ordinary skill in the art based on their general knowledge according to the situation in practical use.

Examples are given below to illustrate various implementations of the present invention, such that the technical solutions disclosed herein can be practiced by one of ordinary skill in the art to which this invention belongs based on the description in the specification. Accordingly, the examples given below are not intended to limit the scope of the claims of the present invention. Moreover, the literatures cited herein are all deemed as being incorporated by reference as part of this specification.

### EXAMPLE 1

### Synthesis of Rhenium-188-Panitumumab Immune Complex

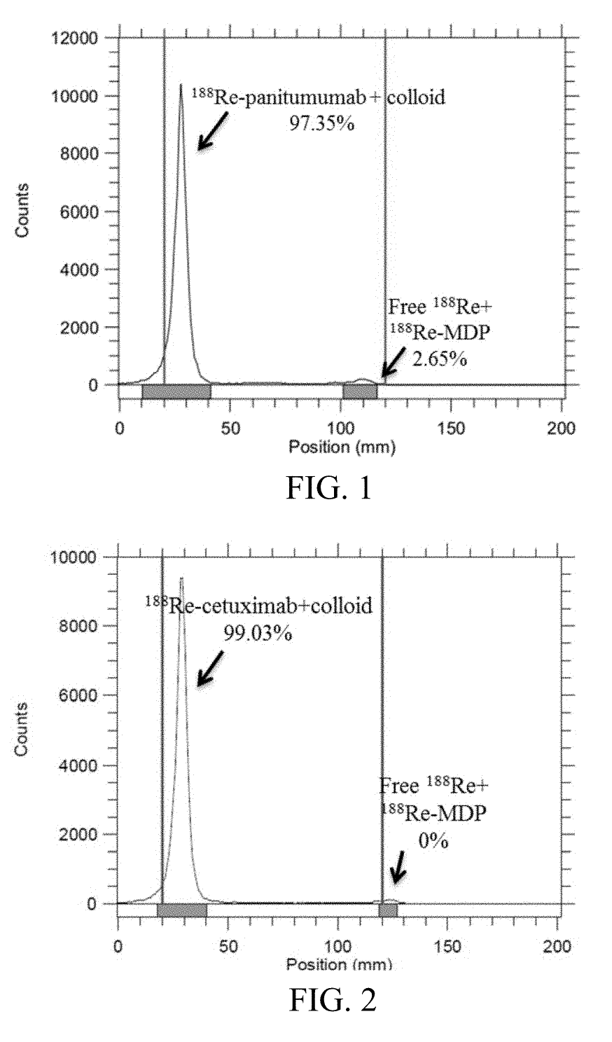

0.2 mg of panitumumab and 2-mercaptoethanol (2-ME) (mAb:2-ME=1:1074) were added to a phosphate buffer to give a total volume of 60 μl, and reacted for 30 min with agitation under room temperature for antibody reduction. Then, excessive 2-ME was removed by centrifuging for 2 min at (2000× g) in a Microspin™ G-50 column, to obtain a purified antibody. The purified antibody was transferred to a sterilized glass bottle, and a bottle of Techne® MDP kit was added and purged with nitrogen for 1 min. Then, 10-20 mCi/400-500 μl of ¹⁸⁸ReO₄⁻ was added, purged with nitrogen for 1 min, and reacted for 8 hrs in a water bath at 37° C. with stirring at 100 rpm, to obtain a rhenium-188-panitumumab immune complex. The radiochemical purity (RCP) was finally analyzed by radio-TLC. The result is shown in FIG. 1. The result shows that the RCP of the rhenium-188-panitumumab immune complex obtained in this example is greater than 90%.

### EXAMPLE 2

### Rhenium-188-Cetuximab Immune Complex

0.2 mg of cetuximab and 2-ME (mAb:2-ME=1:1074) were added to a phosphate buffer to give a total volume of 60 μl, and reacted for 30 min with agitation under room temperature. Then, excessive 2-ME was removed by centrifuging for 2 min at (2000× g) in a Microspin™ G-50 column, to obtain a purified antibody. The purified antibody was transferred to a sterilized glass bottle, and 1.125 mg of methylene diphosphonate (MDP), 0.057 mg of stannous chloride (SnCl₂), and 0.0255 mg of ascorbic acid were added. Then 10 mCi/400 μl of ¹⁸⁸ReO₄⁻ was added, purged with nitrogen for 2 min, and reacted for 4 hrs in a water bath at 37° C. with stirring at 100 rpm, to obtain rhenium-188-cetuximab. The RCP was finally analyzed by radio-TLC. The result is shown in FIG. 2. The result shows that the RCP of the rhenium-188-cetuximab immune complex obtained in this example is greater than 90%.

### EXAMPLE 3

### Preparation of Rhenium-188-Cetuximab as a Kit

5 mg of cetuximab was reduced with 2-ME (mAb:2-ME=1:1074), and the reduced antibody was purified by PD MidiTrap G-25, to remove excessive 2-ME. 1.125 mg of MDP, 0.057 mg of SnCl₂, 0.0255 mg of ascorbic acid, and 0.25 mg of reduced antibody were transferred to a sterilized glass bottle, mixed until uniform, lyophilized for 24 hrs, and then sealed. 10 mCi ¹⁸⁸ReO₄⁻ was added to the sterilized glass bottle, purged with nitrogen for 2 min, reacted for 4 hrs at 37° C. with stirring at 100 rpm, and adjusted with saline to have a suitable activity (50 μCi/70-100 μl).

### EXAMPLE 4

### NanoSPECT/CT Imaging With Rhenium-188-Panitumumab in Xenografted Animal Model of Human Lung Cancer Cells

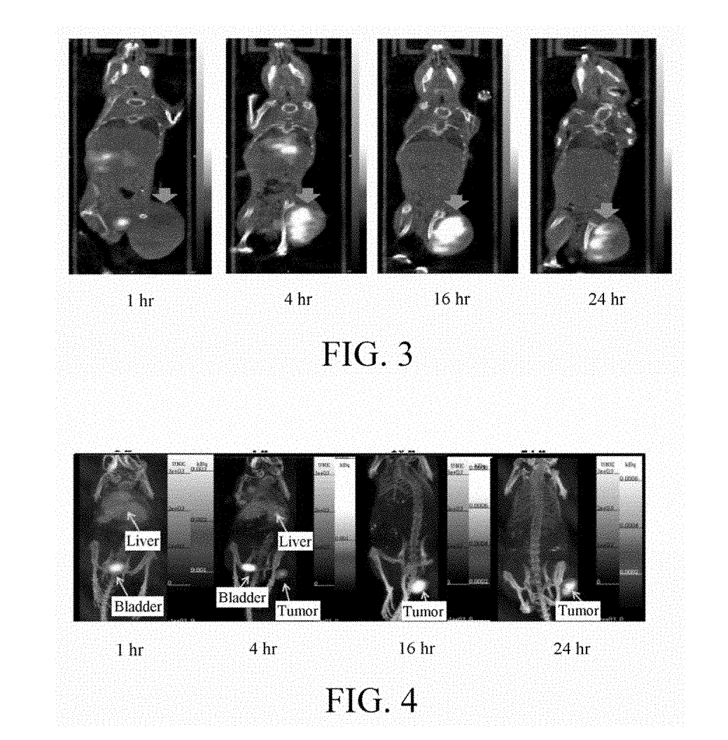

Rhenium-188-panitumumab (50 μCi/70-100 μl) was injected at the tail vein to xenografted animal model of human lung cancer cells NCI-H292, and the mice were anaesthetized with 1-2% of isoflurane (in 100% oxygen) and imaged by nanoSPECT/CT after 1, 4, 16, and 24 hrs. The result is shown in FIG. 3.

As shown in FIG. 3, the antibody is obviously accumulated at the site of tumor 4 hrs after the mice is injected with rhenium-188-panitumumab, and persists for 24 hrs. This suggests that rhenium-188-panitumumab has a specific binding ability to lung cancer cells.

### EXAMPLE 5

### NanoSPECT/CT Imaging With Rhenium-188-Cetuximab in Xenografted Animal Model of Human Lung Cancer Cells and Quantitative Analysis of Activity at the Site of Tumor

Rhenium-188-cetuximab (350 μCi/100 μl) was injected at the tail vein to xenografted animal model of human lung cancer cells NCI-H292, and the mice were anaesthetized with 1-2% of isoflurane (in 100% oxygen) and imaged by nanoSPECT/CT after 1, 4, 16, and 24 hrs. The result is shown in FIG. 4.

As shown in FIG. 4, the antibody is obviously accumulated at the site of tumor 4 hrs after the mice is injected with rhenium-188-cetuximab, and persists for 24 hrs. This suggests that rhenium-188-cetuximab has a specific binding ability to lung cancer cells.

Moreover, the images obtained 24 hrs after the laboratory animals were injected with the agent was circled by using PMOD software, and relevant parameters were calculated from the known activity and image intensity of a reference, to estimate the activity at the site of tumor. The activity was calibrated according to the decay time of the radioactive source, and converted into average tumor uptake (ID/g %) by using the activity at injection, which was respectively 2.94±0.38, 7.32±1.19, 8.43±0.95, and 10.85 (n=1) at 1, 4, 16, and 24 hrs. The experimental result show that the amount of rhenium-188-cetuximab accumulated in the tumor tissue is increased over time.

### EXAMPLE 6

### Rhenium-188-Cetuximab Binds to Epidermal Cell Carcinoma With High Expression of EGFR at High Activity

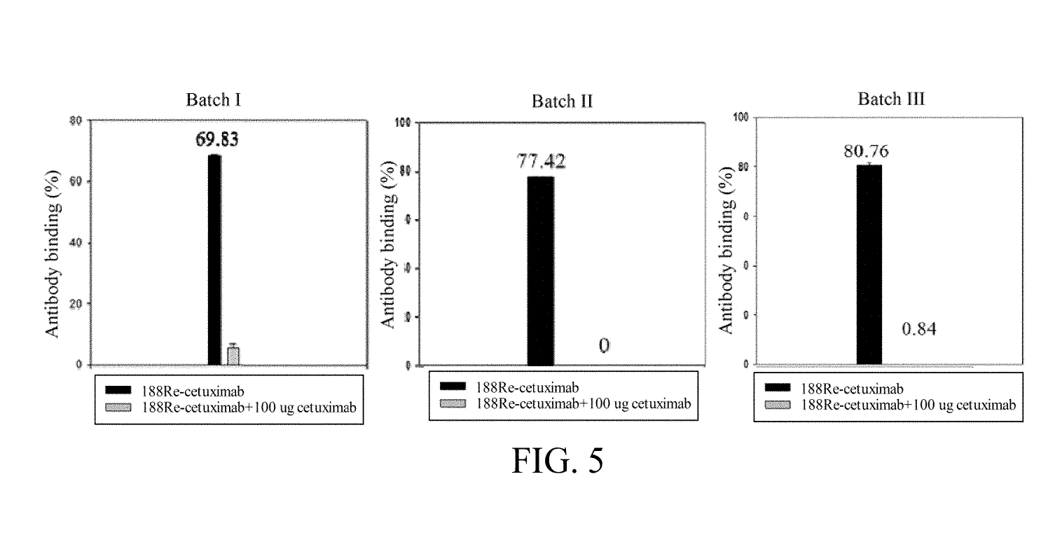

0.5 mL of A431 cells (4×10⁶ cells in each tube) were suspended in a culture medium, part of the cell suspension was added with excessive cetuximab, and the remaining part not. After 30-min-incubation at 37° C., the culture was cooled by standing on ice. 10 μl of diluted rhenium-188-cetuximab was added, and stood for 5 min on ice and then at 37° C. The culture was shaken every 10 min to resuspend the cells, and centrifuged for 1 min at 3000 rcf after 60 min. The activities of the supernatant and the pellet were read on a γ-counter. The result is as shown in FIG. 5, in which “188-Re cetuximab” is rhenium-188-cetuximab, and “cetuximab” is cetuximab without radioactive material labeled. The result shows that the rhenium-188-cetuximab of the present invention has a quite high binding ability to cells with high expression of EGFR.

### EXAMPLE 7

### Cytotoxic Effect of Rhenium-188 and Cetuximab on Human Colorectal Cancer Cells

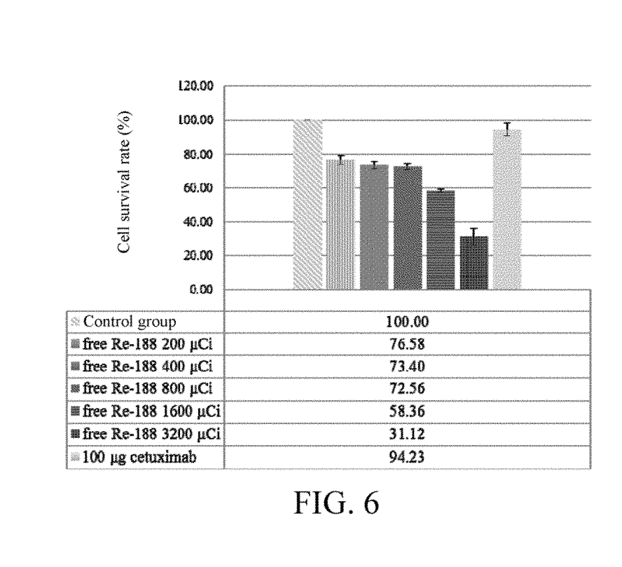

300 μl of HT-29_luc cells were incubated in a 96-well plate (at a density of 1×10⁴ cells per well), and stood overnight at 37° C. The medium was removed, and rinsed with PBS. Rhenium-188 with a suitable activity (0, 200, 400, 800, 1600, and 3200 μCi) and 100 μg of cetuximab as the control were respectively mixed homogeneously with a culture medium (McCoy's medium containing 10% FBS and 1% PS) and added to each well. The incubation was continued for 24 hrs at 37° C. in 5% CO₂. The medium was removed, and the Alamar Blue agent was added and reacted for 4 hrs at 37° C. The fluorescence intensity was detected (at an excitation wavelength of 535 nm/emission wavelength of 595 nm). The result is shown in FIG. 6. As shown in FIG. 6, in the presence of rhenium-188, the viability of colorectal cancer cells is low, and the cytotoxic effect is better than that of cetuximab given alone.

### EXAMPLE 8

### In-Vitro Stability Analysis of Rhenium-188-Cetuximab

Rhenium-188-cetuximab products (500-600 μl) stored in phosphate buffer solution (PBS) were respectively stood at 4° C. and room temperature, and evaluated for the stability by radio-TLC. Furthermore, 10 μl of the rhenium-188-cetuximab product was added to 190 μl of rat serum. After 0, 1, 4, and 24 hrs, 10 μl was transferred to a trichloroacetic acid (TCA) solution, stood for 15 min on ice, and passed through a 0.45 μm filter membrane. The activities before filtration and at various time points after filtration were read, and the rate of the radioactive isotope binding to the antibody was calculated according to the formula (activity before filtration-activity after filtration)/activity before filtration×100, to evaluate the drug stability. The result is shown in Table 1. It can be known from the result in Table 1 that the rate of the radioactive isotope binding to the antibody in the antibody product is still maintained at about 92.54% after standing for 24 hrs at 4° C., and at about 89.86% at room temperature (RT). Moreover, it is found through biopsy that the rhenium-188-cetuximab of the present invention has a good stability in rat serum, about 89.47% was retained after 4 hrs, and about 76.84% was retained after 24 hrs.

TABLE 1
In-vitro stability of rhenium-188-cetuximab

| | | |
|---|---|---|
| Incubation | PBS (%) | Rat serum (%) |

| time (h) | 4° C. | RT | 37° C. |
|---|---|---|---|
| 0 | 95.04 ± 1.29 | 94.84 ± 1.39 | 97.86 ± 0.44 |
| 1 | 95.12 ± 1.96 | 95.70 ± 2.52 | 93.69 ± 0.61 |
| 4 | 96.64 ± 0.29 | 94.12 ± 1.25 | 89.47 ± 0.50 |
| 24 | 92.54 ± 5.17 | 89.86 ± 3.36 | 76.84 ± 1.51 |

It can be known from the result in the examples above that the radioimmune complex of the present invention has a high affinity and specificity to cancers with high expression of EGFR, is useful as an agent for diagnosing and treating tumors with high expression of EGFR, and has a good stability. The present invention can facilitate the development of nuclear imaging in tumor detection or treatment.

The specific examples disclosed above are not intended to limit the scope of the claims of the present invention. Modifications may be made by those of ordinary skill in the art based on their general knowledge without departing from the principle and spirit of the present invention, and thus the scope of the present invention is defined by the claims.

What is claimed is:

1. A radioimmune complex, comprising:
an epidermal growth factor receptor (EGFR)-targeted antibody that is cetuximab or panitumumab; and
a radioactive isotope of rhenium labeled thereon.

2. The radioimmune complex according to claim 1, wherein the radioactive isotope of rhenium is rhenium-188 or rhenium-186.

3. The radioimmune complex according to claim 1, wherein the EGFR-targeted antibody is reduced with 2-mercaptoethanol.

4. The radioimmune complex according to claim 1, prepared through a process comprising
a) treating the EGFR-targeted antibody with 2-mercaptoethanol, to obtain a reduced EGFR-targeted antibody;
b) adding a complexing agent to the reduced EGFR-targeted antibody; and
c) labeling the EGFR-targeted antibody with a radioactive isotope of rhenium.

5. The radioimmune complex according to claim 4, wherein the process further comprises adding a reducing agent and a stabilizing agent to the reduced EGFR-targeted antibody.

6. The radioimmune complex according to claim 5, wherein the EGFR-targeted antibody is cetuximab.

7. The radioimmune complex according to claim 4, wherein the complexing agent is methylene diphosphonate (MDP).

8. The radioimmune complex according to claim 5, wherein the reducing agent is stannous chloride.

9. The radioimmune complex according to claim 5, wherein the stabilizing agent is ascorbic acid.

10. A radioactive theranostic agent, comprising:
a radioimmune complex, comprising
an epidermal growth factor receptor (EGFR)-targeted antibody that is cetuximab or panitumumab; and
a radioactive isotope of rhenium labeled thereon; and
a theranostically acceptable excipient.

11. A kit, comprising:
an epidermal growth factor receptor (EGFR)-targeted antibody that is cetuximab or panitumumab;
2-mercaptoethanol; and
a complexing agent.

12. The kit according to claim 11, wherein the complexing agent is methylene diphosphonate.

13. The kit according to claim 11, further comprising a reducing agent and a stabilizing agent.

14. The kit according to claim 12, wherein the reducing agent is stannous chloride, and the stabilizing agent is ascorbic acid.
